(19) United States
(12) Patent Application Publication
(10) Pub. No.: US 2026/0240676 A1
(43) Pub. Date: Aug. 20, 2026

(54) OUTLET CLOSURE AND DRAINING AND SECUREMENT SYSTEM FOR DRAINABLE POUCH

(71) Applicant: HOLLISTER INCORPORATED, Libertyville, IL (US)

(72) Inventor: Robert J. Weinberg, Lake Villa, IL (US)

(73) Assignee: HOLLISTER INCORPORATED, Libertyville, IL (US)

(21) Appl. No.: 18/881,939

(22) PCT Filed: Jul. 19, 2023
(86) PCT No.: PCT/US2023/070485

§ 371 Date: Jan. 7, 2025

Related U.S. Application Data
(60) Provisional application No. 63/391,135, filed on Jul. 21, 2022.

(51) Int. Cl. A61F 5/44; A61F 5/445

(52) U.S. Cl. CPC A61F 5/4407; A61F 5/445

(57) ABSTRACT

A drainable pouch including a body side wall, a distal wall, a downwardly extending portion, a closure member, and at least one cover is provided. The body side wall and distal wall may be joined along a portion of their peripheral edges to define a cavity therebetween for collecting body waste. The downwardly extending outlet portion may terminate in a discharge opening for draining. The closure member may be disposed at an end of the outlet portion, the closure member having a plurality of foldable members foldable upwardly toward the cavity to close the outlet portion, one or more of the foldable members having outwardly extending tabs, ends of the plurality of foldable members being flexible inwardly to open the outlet portion. The at least one cover may be attached to one of the body side wall and distal side wall to overlie the plurality of foldable members.

### BACKGROUND

The present disclosure pertains to a drainable pouch. More particularly, the present disclosure pertains to a system for draining, closing, and securing a drainable pouch.

Ostomy pouches for collecting body waste are used by patients who have had surgery such as a colostomy, ileostomy, or urostomy. Ostomy pouches typically include flat, opposing side walls secured together along their edges to define a collection cavity. One of the side walls is provided with an opening to receive a stoma, and means to secure the pouch to the user, such as an adhesive barrier, so that body waste discharged through the stoma is received within the cavity.

The ostomy pouch may be a closed-end pouch for a single use, in which case the entire pouch is discarded after it has been substantially filled with stomal discharge. Alternatively, the ostomy pouch can be a drainable pouch with a discharge opening at its lower end, which may be closed during collection of body waste material, but may be opened for draining body waste material from the pouch after a period of use. The discharge opening of drainable pouches is typically defined at the end of a narrowed outlet portion, which is provided with closure means for maintaining the discharge opening in a sealed condition until waste material is to be drained from the pouch.

For quality of life of the users, drainable pouches should be easy to drain without risking soiling of clothes or the surroundings. They also should be easy to close securely after being drained and amenable to being cleaned after drainage and before closing again, such that the risk of unpleasant odor is substantially reduced. Most importantly, the closure means should provide a secure seal when closed to minimize the risk of leakage. Further, it is also desirable that the closure means provide discretion for users.

Accordingly, there is a need for an improved system to drain, close, and secure a drainable pouch outlet.

### BRIEF SUMMARY

An outlet closure and a draining and securement system for a drainable pouch is provided according to various embodiments. The outlet closure may include at least one tab and at least one cover to drain, close, and secure an outlet portion of the drainable pouch.

In one aspect, a drainable pouch may include a body side wall and a distal wall joined along a portion of their peripheral edges to define a cavity therebetween for collecting body waste, a downwardly extending outlet portion terminating in a discharge opening for draining body waste collected in the cavity, a closure member disposed at an end of the outlet portion, and at least one cover attached to one of the body side wall and distal side wall to overlie the plurality of foldable members when the foldable members are folded upwardly toward the cavity in a closed position. The closure member may have a plurality of foldable members foldable upwardly toward the cavity to close the outlet portion, one or more of the foldable members may have outwardly extending tabs, ends of the plurality of foldable members may be flexible inwardly to open the outlet portion.

In an embodiment, the extending tabs may include two tabs that extend past opposite outer edges of the outlet portion. The two tabs may extend from opposite sides of the outlet portion transverse to the outlet portion. The two tabs may be configured to aid in opening the outlet portion. The two tabs may be located near the discharge opening.

In an embodiment, the at least one cover may include a first cover and a second cover. The first cover may be attached to the distal wall and the second cover is attached to the body side wall. The first and second cover may include a first and second fastener. The first and second covers may secure the closure member when the closure member is in a closed position and the first and second cover members are fastened to one another.

In an embodiment, the at least one cover may cover the outlet portion and at least one tab.

In an embodiment, the closure member may include at least one transversely-extending fold-line configured to facilitate folding up the closure member. The closure member may further include one or more vertically-extending creases and/or angled creases to facilitate opening the closure member and outlet portion to empty the drainable pouch.

In another aspect, a drainable pouch may include a body side wall and a distal wall joined along a portion of their peripheral edges to define a cavity therebetween for collecting body waste, a downwardly extending outlet portion terminating in a discharge opening for draining body waste collected in the cavity, and a closure member attached to the outlet portion. The closure member may include a plurality of foldable members foldable upwardly toward the cavity to close the outlet portion. One of the foldable members may include two tabs extending transversely past opposite outer edges of the outlet portion. The tabs may be configured to aid in opening the outlet portion. The tabs may be located near the discharge opening.

In an embodiment, the closure member may include at least one transversely-extending fold-line configured to facilitate folding up the closure member and at least one vertically-extending and/or angled fold line configured to facilitate opening of the outlet portion. The at least one transversely-extending fold-line and the at least one vertically-extending and/or angled fold line may be configured to facilitate bi-directional manipulation of the closure member.

In some embodiments, the drainable pouch may further include at least one cover attached to one of the body side wall and distal side wall to overlie the outlet portion when the outlet portion is folded up in a closed position. The at least one cover may be configured to cover the two tabs when the outlet portion is folded up in a closed position. For example, the drainable pouch may include a first cover and a second cover, wherein the first cover may be attached to the distal wall and the second cover may be attached to the body side wall. The first and second covers may include a first fastener and a second fastener to secure the first and second covers to one another.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is body side view of an outlet portion of an ostomy pouch in an open position, according to an embodiment.

FIG. 2 is a distal side view of the outlet portion of the ostomy pouch of FIG. 1 in an open position.

FIG. 3 is a distal side view of the outlet portion of the ostomy pouch of FIG. 1 in a closed position.

FIG. 4A is a schematic cross-sectional view of the outlet portion of the ostomy pouch of FIG. 1 in an open position.

FIG. 4B is a schematic cross-sectional view of the outlet portion of the ostomy pouch of FIG. 1 folded up one time.

FIG. 4C is a schematic cross-sectional view of the outlet portion of the ostomy pouch of FIG. 1 folded up two times.

FIG. 4D is a schematic cross-sectional view of the outlet portion of the ostomy pouch of FIG. 1 folded up three times.

FIG. 4E is a schematic cross-sectional view of the outlet portion of the ostomy pouch of FIG. 1 folded up three times and secured in place with a pair of connected covers.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular.

The present disclosure provides a drainable ostomy pouch that includes an outlet closure and a draining and securement system. The outlet closure may include at least one tab and at least one cover for securely and easily draining, closing, and securing the outlet portion of the drainable pouch.

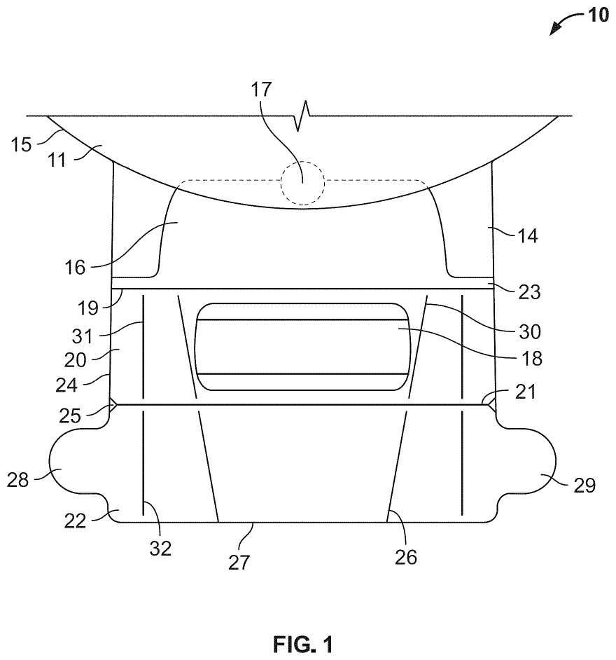

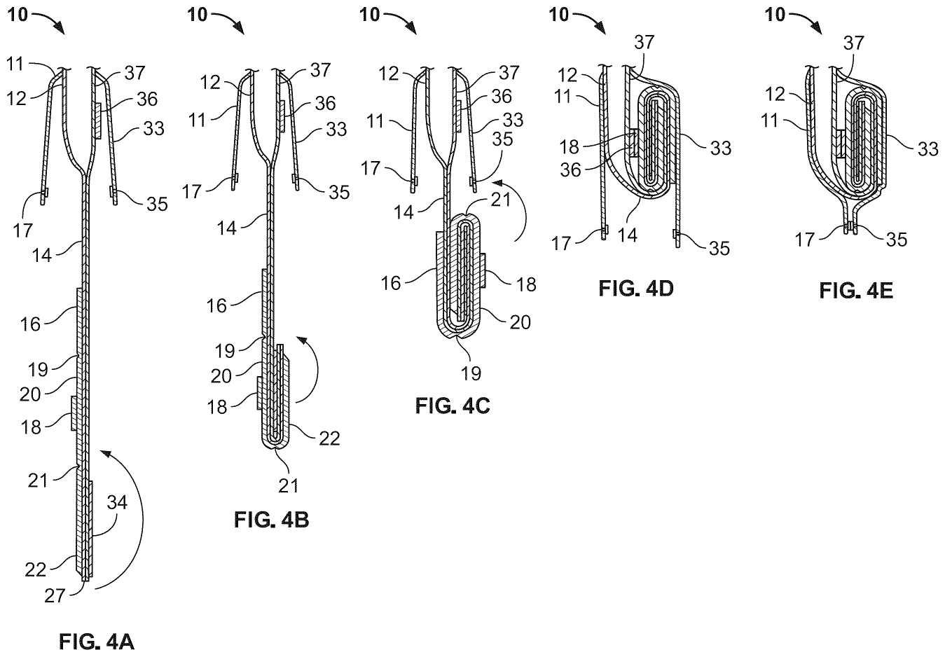

Referring now to the figures, FIGS. 1-4E show a drainable pouch 10 according to an embodiment. The drainable pouch 10 includes a distal wall 37 and a body side wall 12 (FIG. 4A), which may be joined along their peripheral edges 15 (FIG. 3) to define a cavity therebetween for collecting stomal discharge. The drainable pouch 10 may include a downwardly extending outlet portion 14 terminating in a discharge opening 27 for draining the contents collected in the cavity after a period of use. The discharge opening 27 of drainable pouch 10 may be closed during use by folding outlet portion 14 upwardly and securing it in the upwardly folded position as shown in FIGS. 3 and 4E. The drainable pouch 10, for example, may be a drainable ostomy pouch.

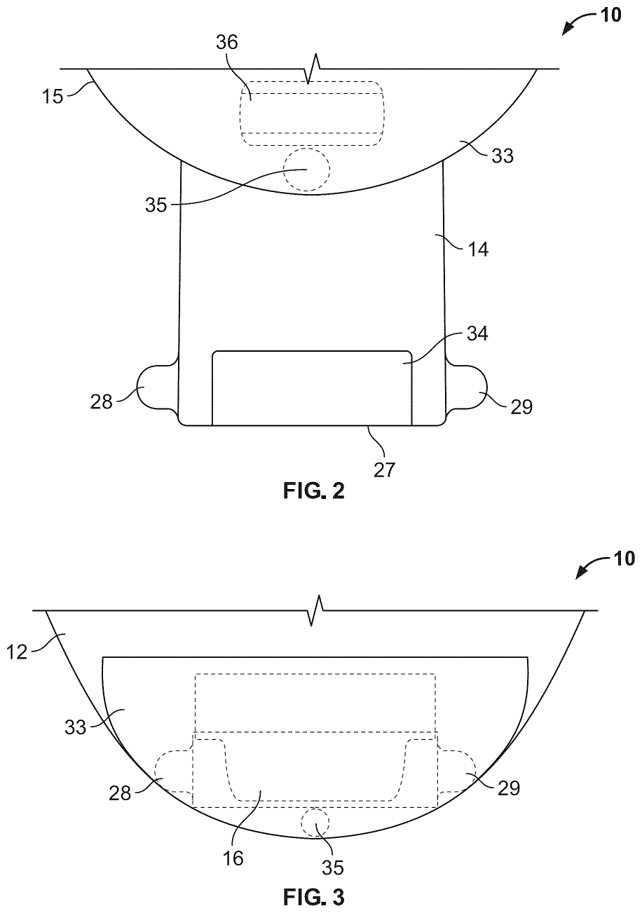

FIGS. 1 and 2 show a body side view and a distal side view, respectively, of an outlet portion of drainable ostomy pouch 10 in an open position. The drainable pouch 10 generally includes an internal collection portion or cavity 13 (FIG. 4A) for the collection of stomal discharge, an outlet portion 14 having a discharge opening 27, and one or more covers 11, 33, that can include fasteners 17, 35. A closure member 24 is mounted to the pouch 10 at the outlet portion 14. A first fastener strip 18 is mounted to or formed integrally with the closure member 24. A second fastening strip 36 may be mounted to the pouch 10 on the pouch distal side wall 37, as will be described in more detail below. A stiffening member 34 can be mounted to the pouch distal side wall 37 at the outlet portion 14, opposite the closure member 24, and discharge opening 27.

In an embodiment, the closure member 24 is formed as a single-piece member including a first fold section 22, a second fold section 20, and a third fold section 16. In an embodiment, a portion 23 of the third fold section 16 extends fully across the width of the outlet portion 14, whereas another portion 38 of the third fold section does not extend fully across the outlet portion 14 width. In such an embodiment, the closure member 24 includes a first transversely extending fold line 21 defined between the first and second fold sections 22, 20 and a second transversely extending fold line 19 defined between the second fold section 20 and the third fold section 16, which are configured to facilitate folding up and closing of outlet portion 14. The first and second transversely extending fold lines 19, 21 allow the single-piece closure member to have a liquid tight seal. The closure member 24 may also include vertically extending and angled fold lines 26, 30, 31, 32 configured to facilitate opening the discharge opening 27 by allowing ends of the closure member 24 to be bent inwardly toward one another at the fold lines 26, 30, 31, 32. In an embodiment, fold lines 31, 32 bend inwardly and fold lines 26, 30 bend outwardly.

In the embodiment of FIG. 1, the closure member 24 includes tabs 28, 29 configured to provide gripping points for opening and closing the closure member 24. In this embodiment, the tabs 28, 29 are integrally formed with the closure member 24 extending outwardly from the first fold section 22. In other embodiments, the tabs 28, 29 may be formed with the closure member 24 extending outwardly from the second fold section 20 or the third fold section 16. In other embodiments, the tabs 28, 29 may be separately formed from the closure member 24. In other embodiments, the tabs 28, 29 may be formed on or integral with the stiffening member 34.

The closure member 24 may be formed from a suitable material, such as a polymeric material. The closure member 24 may have a thickness of about 2 mil to about 40 mil, preferably about 5 mil to about 20 mil, and more preferably about 8 mil to about 15 mil. In an embodiment, the tabs 28, 29 may be made out of the same material as the closure member 24. The closure member 24 may be formed from a suitably flexible material to allow bending (See, for example, FIGS. 4B through 4D), but with sufficient stiffness (relative to the pouch 10 material) to maintain shape in the unfolded state.

The first transversely-extending fold-line 21 may be arranged between the first fold section 22 and the second fold section 20 and may be configured to allow the outlet portion 14 to be folded up such that the first fold section 22 is positioned on the distal side of the outlet portion 14 after a first fold as shown in FIG. 4B. The second transversely-extending fold-line 19 may be formed between the second fold section 20 and the third fold section 16 and may be configured to allow the outlet portion 14 to be folded up such that the first and second fold sections 22, 20 are positioned on the distal side of the outlet portion 14 after a second fold as shown in FIG. 4C.

The closure member 24, includes the transversely-extending fold-lines 19, 21 to permit the closure member 24 and pouch outlet 14 to be folded up and down (to open and close the outlet 14), and includes the vertically extending and angled fold lines 26, 30, 31, 32 to permit opening and closing the pouch outlet 14 and discharge opening 27, thus providing bi-directional manipulation of the closure 24 member.

The transversely-extending fold lines 19, 21 and the axially-extending fold lines 26, 30, 31, 32 may be defined by creases, shallow cuts or embossments in the closure member 24. For example, the fold lines may be provided by cutting into the closure member 24 via any known method, such as via laser cutting or die cutting. Alternatively, the fold lines may be defined by living hinges formed during a molding or extrusion process of the one-piece closure member 24, each of which may comprise creases on both the distal surface and pouch-facing surface of the closure member 24. In some embodiments, the closure member 24 may include three separately formed fold members 22, 20, 16, wherein the transversely-extending fold-lines 19, 21 may be defined by spaces between the fold members 20, 22, 16.

In an embodiment, the outlet portion 14 may include cut-outs 25 located between the first and second fold section 22, 20. The cut-outs 25 soften the corners of the folded outlet and enhance flexibility for opening and closing the outlet potion 14.

As shown in FIGS. 1 and 2, the drainable pouch 10 may include covers 11, 33 including fasteners 17, 35. The covers 11, 33 may be formed from a suitable material, such as non-woven, fabric, polymeric materials and the like. In an embodiment, the covers 11, 33 may be formed from a non-woven or fabric material of a desired color to provide discretion when the outlet portion 14 is folded up and secured under the covers 11, 33, as shown in FIGS. 3 and 4E. In another embodiment, one or both of the covers 11, 33 may be made out of a translucent material to allow the user to see that the outlet portion 14 is closed and secured under the covers 11, 33.

In an embodiment, the stiffening member 34 provides a measure of stiffness to the outlet portion 14 to enhance opening and maintaining the outlet portion open to, for example, empty the contents of the pouch 10. The stiffening member 3 may be made out of the same material as or a different material from the closure member 24.

FIG. 3 shows a distal side view of the outlet portion 14 of the drainable pouch 10 of FIG. 1 in a closed position. In an embodiment, the tabs 28, 29 are entirely covered by the one or both of the covers 11, 33, while in a closed position. In another embodiment, when the outlet portion 14 is rolled up and sealed under the covers 11, 33, the entirety of the closure member 24 and the tabs 28, 29 are covered by the covers 11, 33.

FIG. 4A shows a schematic cross-sectional view of an embodiment of the pouch 10, with the outlet portion 14 in an open position. The drainable pouch 10 may be configured to be closed by folding up the outlet portion 14 toward the distal side wall 37 and connecting the fastener strips 18, 36, as shown in FIG. 4D. The fastener strips 18, 36 and the fasteners 17, 35 may include fastener elements, which are adapted for releasable interlocking engagement. The drainable pouch 10 may be secured by connecting the fasteners 17, 35, as shown in FIG. 4E.

In another embodiment, the drainable pouch 10 may have the cover 33 and may be secured by connecting the fastener 35 to a fastener on the body side wall 12. In another embodiment, the drainable pouch 10 may have the cover 33 and may be secured be secured behind the cover 33.

To close the outlet portion 14, the outlet portion 14 may be folded toward a distal surface of the drainable pouch 10 along the transversely-extending fold-lines 19, 21. In this embodiment, the drainable pouch 10 may be configured to be closed and hidden by folding up the outlet portion 14 three times, the connecting fastener strips 18, 36, the concealing covers 11, 33, and the connecting fasteners 17, 35, thereby securing the outlet portion 14 in the closed and hidden position, as shown in FIGS. 4A-4E. In other embodiments, the drainable pouch 10 may be configured to be closed after folding up the outlet portion 14 once or twice or more than three times.

In an embodiment, the tabs 28, 29 are configured to aid in opening, closing, folding up, folding down, sealing, and securing the outlet portion 14. In this embodiment, the tabs 28, 29 are positioned near the first fold member 22 and extend beyond the outer edges of the outlet portion 14 to provide a location distanced or spaced from the discharge opening for a user's hand or fingers to assist in opening the discharge opening 27. For example, the tabs 28, 29 may be used to move the sides of the outlet portion 14 together to easily open the drainage pathway of the discharge opening 27. In another example, the tabs 28, 29 may be pulled in opposing directions to close the discharge opening 27.

In an embodiment, the tabs 28, 29 are configured to provide gripping points for emptying the drainable pouch 10 through the discharge opening 27 by gripping and pushing the tabs 28, 29 together.

In an embodiment, the fold lines 26, 30, 31, 32 may be located in the first and second fold members 22, 20. In this embodiment, the fold lines 26, 30, 31, 32 may coincide with the inside edges of a heat seal defined in the inner width of the outlet portion 14 to provide a flexible line to aid in the drain flow path of the discharge opening 27.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A drainable pouch, comprising:
a body side wall and a distal wall joined along a portion of their peripheral edges to define a cavity therebetween for collecting body waste;
a downwardly extending outlet portion terminating in a discharge opening for draining body waste collected in the cavity;
a closure member disposed at an end of the outlet portion, the closure member having a plurality of foldable members foldable upwardly toward the cavity to close the outlet portion, one or more of the foldable members having outwardly extending tabs, ends of the plurality of foldable members being flexible inwardly to open the outlet portion; and
at least one cover attached to one of the body side wall and distal side wall to overlie the plurality of foldable members when the foldable members are folded upwardly toward the cavity in a closed position.

2. The drainable pouch of claim 1, wherein the extending tabs comprise two tabs that extend past opposite outer edges of the outlet portion.

3. The drainable pouch of claim 2, wherein the two tabs extend from opposite sides of the outlet portion transverse to the outlet portion.

4. The drainable pouch of claim 2, wherein the two tabs are configured to aid in opening the outlet portion.

5. The drainable pouch of claim 2, wherein the two tabs are located near the discharge opening.

6. The drainable pouch of claim 2, wherein the at least one cover comprises a first cover and a second cover, wherein the first cover is attached to the distal wall and the second cover is attached to the body side wall.

7. The drainable pouch of claim 2, wherein the first and second covers include a first fastener and a second fastener to secure the first and second covers to one another.

8. The drainable pouch of claim 7, wherein the first and second covers secure the closure member when the closure member is in a closed position and the first and second cover members are fastened to one another.

9. The drainable pouch of claim 1, wherein the at least one cover covers the outlet portion and at least one tab.

10. The drainable pouch of claim 1, wherein the closure member includes at least one transversely-extending fold-line configured to facilitate folding up the closure member.

11. The drainable pouch of claim 1, wherein the closure member further includes one or more vertically-extending fold lines and/or angled fold lines to facilitate opening the closure member and outlet portion to empty the drainable pouch.

12. The drainable pouch of claim 1, wherein the closure member includes at least one transversely-extending fold-line configured to facilitate folding up the closure member and at least one vertically-extending and/or angled fold line configured to facilitate opening of the outlet portion, wherein the at least one transversely-extending fold-line and the at least one vertically-extending and/or angled fold line are configured to facilitate bi-directional manipulation of the closure member.

13. A drainable pouch, comprising:
a body side wall and a distal wall joined along a portion of their peripheral edges to define a cavity therebetween for collecting body waste;
a downwardly extending outlet portion terminating in a discharge opening for draining body waste collected in the cavity; and
a closure member attached to the outlet portion, the closure member including a plurality of foldable members foldable upwardly toward the cavity to close the outlet portion, wherein one of the foldable members includes two tabs extending transversely past opposite outer edges of the outlet portion and configured to aid in opening the outlet portion.

14. The drainable pouch of claim 13, the closure member includes at least one transversely-extending fold-line configured to facilitate folding up the closure member and at least one vertically-extending and/or angled fold line configured to facilitate opening of the outlet portion, wherein the at least one transversely-extending fold-line and the at least one vertically-extending and/or angled fold line are configured to facilitate bi-directional manipulation of the closure member.

15. The drainable pouch of claim 13, wherein the tabs are located near the discharge opening.

16. The drainable pouch of claim 13, further including at least one cover attached to one of the body side wall and distal side wall to overlie the outlet portion when the outlet portion is folded up in a closed position.

17. The drainable pouch of claim 16, wherein the at least one cover comprises a first cover and a second cover, wherein the first cover is attached to the distal wall and the second cover is attached to the body side wall, wherein the first and second covers include a first fastener and a second fastener to secure the first and second covers to one another.

18. The drainable pouch of claim 16, wherein the at least one cover covers the two tabs when the outlet portion is folded up in the closed position.
